(19) United States
(12) Patent Application Publication
(10) Pub. No.: US 2024/0339662 A1
(43) Pub. Date: Oct. 10, 2024

(54) FUNCTIONALIZED CYCLIC ETHERS FOR LITHIUM-ION BATTERIES

(71) Applicant: NOHMS Technologies, Inc., Rochester, NY (US)

(72) Inventors: Gabriel Torres, Rochester, NY (US); John Sinicropi, Rochester, NY (US); Dai in Park, Daejeon City (KR); Surya S. Moganty, Henrietta, NY (US)

(73) Assignee: NOHMS Technologies, Inc., Rochester, NY (US)

(21) Appl. No.: 18/624,612

(22) Filed: Apr. 2, 2024

Related U.S. Application Data
(60) Provisional application No. 63/456,889, filed on Apr. 4, 2023.

(51) Int. Cl. H01M 10/0567; C07D 303/22; C07F 7/18; C07F 9/655; H01G 11/64; H01M 4/02; H01M 4/134; H01M 4/36; H01M 4/38; H01M 4/62; H01M 10/0525

(52) U.S. Cl. CPC H01M 10/0567; C07D 303/22; C07F 7/188; C07F 9/6551; C07F 9/65515; C07F 9/6552; H01G 11/64; H01M 4/134; H01M 4/366; H01M 4/386; H01M 4/624; H01M 10/0525; H01M 2004/027

(57) ABSTRACT

The present disclosure relates to a functionalized cyclic ether that is useful for stable cycling, storage of lithium-ion cells at high temperatures, and performance at low temperatures, an electrolyte containing the functionalized cyclic ether, and an electrochemical energy storage device containing the electrolyte.

### CROSS-REFERENCE

This application is related to and claims priority to U.S. Provisional Patent Application No. 63/456,889 filed Apr. 4, 2023, which is incorporated herein by reference.

### FIELD

The present disclosure relates to functionalized cyclic ethers that are useful for reducing battery resistance, increasing cycle life, improving high-temperature performance; an electrolyte containing the functionalized cyclic ethers, and electrochemical energy storage devices containing these electrolytes.

Li-ion batteries are heavily used in consumer electronics, electric vehicles (EVs), as well as energy storage systems (ESS) and smart grids. Recently, Li-ion batteries with voltages above 4.35 V have gained importance because of higher capacity and subsequent energy density benefits. However, the stability of the cathode materials at these potentials reduces due to increased oxidation. This may result in electrochemical oxidation of the material to produce gases, and that can deteriorate the performance of the battery. The cathode active material, which is capable of intercalating/deintercalating lithium ions may dissolve in the non-aqueous electrolyte, resulting in a structural breakdown of the material, and will lead to an increase in the interfacial resistance. These Li-ion batteries are also typically exposed to extreme temperatures during their operation. The SEI (Solid Electrolyte Interface) layer formed on the anode is gradually broken down at high temperatures, and hence leads to more irreversible reaction resulting in capacity loss. Similarly, the CEI (Cathode Electrolyte Interface) will also lose stability at elevated temperatures. These reactions happen on the positive and negative electrode during cycling but are generally more severe at higher temperatures due to faster kinetics. The next generation Li-ion batteries used in consumer electronics, EVs, and ESS will require significant improvements in the electrolyte component relative to the current state-of-the art of Li-ion batteries.

The shuttling of positive and negative ions between the battery electrodes is the main function of the electrolyte. Historically, researchers have focused on developing battery electrodes, and electrolyte development has been limited. Traditional Li-ion batteries used carbonate-based electrolytes with a large electrochemical window, which can transport lithium ions. These electrolytes need functional additives to passivate the anode and form a stable SEI, as well as additives for stabilizing the cathode. At the same time, there is a need to design and develop compounds that allow stable and safe cycling of high voltage, high energy Li-ion batteries.

As the industry moves towards higher energy cathode materials for higher energy batteries, stable, efficient, and safe cycling of batteries in wide voltage windows is necessary. Li-ion battery electrolytes can be tuned based on their applications by addition of different co-solvents and additives. This tunability has enabled the development of different additives for high voltage stability and safety of Li-ion cells.

There have been reports in the literature of cyclic ethers in Li-ion batteries. U.S. Pat. No. 9,640,839 has reported the use of diglycidyl oxalate as a component of an electrolyte for lithium secondary batteries. U.S. Pat. No. 9,153,820 discloses a family of nitrile functionalized oxolanyl and oxepanyl compounds for lithium secondary batteries. U.S. Pat. No. 10,998,580 discloses 2-[(methylsulfonyl)methyl]-oxirane as an additive that decreases the impedance in a lithium battery.

Hence, there is a need to develop and incorporate novel compounds to improve the performance of lithium-ion batteries.

### SUMMARY

Herein, functionalized cyclic ethers are reported for use in Li-ion batteries. These molecules when added to electrolytes allow for stabilization of the cathode and the holistic electrolyte system. A cell with functionalized cyclic ethers in the electrolyte would enable safe, long cycle life, and high energy lithium-ion batteries.

In accordance with one aspect of the present disclosure, there is provided a new class of compounds, and an electrolyte for an electrochemical energy storage device. The electrolyte includes: a functionalized cyclic ether; an aprotic organic solvent system; and a metal salt.

In accordance with another aspect of the present disclosure, there is provided an electrolyte for an electrochemical energy storage device, the electrolyte includes: a functionalized cyclic ether; an aprotic organic solvent system; a metal salt; and at least one additive.

In accordance with another aspect of the present disclosure, there is provided an electrochemical energy storage device, including: a cathode; an anode; a separator and an electrolyte including a functionalized cyclic ether, an aprotic organic solvent system, and a metal salt.

In accordance with another aspect of the present disclosure, there is provided an electrolyte for an electrochemical energy storage device, the electrolyte includes: a functionalized cyclic ether; an aprotic organic solvent system; a metal salt; and at least one additive, wherein the aprotic organic solvent includes open-chain or cyclic carbonate, carboxylic acid ester, nitrite, ether, sulfone, sulfoxide, ketone, lactone, dioxolane, glyme, crown ether, siloxane, phosphoric acid ester, phosphite, mono- or polyphosphazene or mixtures thereof.

In accordance with another aspect of the present disclosure, there is provided an electrolyte for an electrochemical energy storage device, the electrolyte includes: a functionalized cyclic ether; an aprotic organic solvent system; a metal salt; and at least one additive, wherein the cation of the metal salt is aluminum, magnesium or an alkali metal, such as lithium or sodium.

In accordance with another aspect of the present disclosure, there is provided an electrolyte for an electrochemical energy storage device, the electrolyte includes: a functionalized cyclic ether; an aprotic organic solvent system; a metal salt; and at least one additive, wherein the additive contains a compound containing at least one unsaturated carbon-carbon bond, carboxylic acid anhydride, sulfur-containing compound, phosphorus-containing compounds boron-containing compound, silicon-containing compound or mixtures thereof.

These and other aspects of the present disclosure will become apparent upon a review of the following detailed description and the claims appended thereto.

### DETAILED DESCRIPTION

The disclosed technology relates generally to lithium-ion (Li-ion) battery electrolytes. Particularly, the disclosure is directed towards functionalized cyclic ethers covalently bonded to either at least one phosphite group, a nitrile group, a silyl group, or a sulfonate group; electrolytes containing these functionalized cyclic ether materials; and electrochemical energy storage devices containing these electrolytes.

The present disclosure describes a Li-ion battery electrolyte with a formulation that can overcome cathode stability challenges in Li-ion batteries, particularly those including cathode materials that operate at high voltage. Current state-of-the-art Li-ion batteries include cathode materials that are low in nickel content and operate at high voltage or have high nickel content but operate at a low voltage. State-of-the-art electrolytes are tuned towards these conditions, and researchers have recently started focusing on enabling high nickel, high voltage battery cathodes with novel electrolyte compositions. There is a need to develop an electrolyte solution for cycling of Li-ion cells with high voltage, high nickel cathodes. The present technology is based on an innovative material including a functionalized cyclic ether, which can improve the stability of high-voltage, high-energy cathodes. These electrolyte components form a unique cathode electrolyte interface (CEI) and do not excessively passivate the cathode, when used at low weight loadings. An improved CEI improves the high temperature performance and storage stability.

In an embodiment, an electrochemical energy storage device electrolyte includes a) an aprotic organic solvent system; b) a metal salt; c) a functionalized cyclic ether and d) at least one additive.

In an aspect of the disclosure, the molecular structure of the functionalized cyclic ethers covalently bonded to either at least one phosphite group, a nitrile group, a silyl group, or a sulfonate group are selected from a list consisting of oxiranes, oxetanes, oxanes, and oxipanes.

In an aspect of the disclosure, the functionalized cyclic ethers have a molecular structure according to formulas I, II, III or IV:

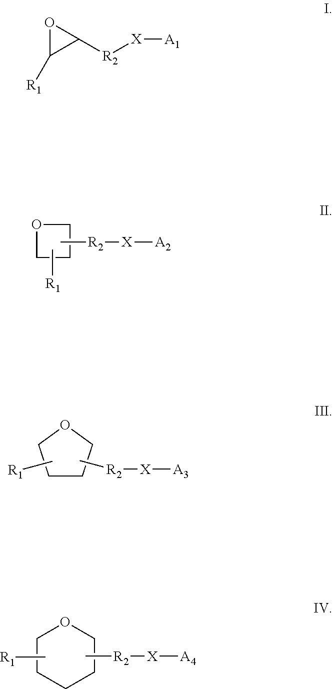

wherein:
R₁ is a hydrogen, halogen, C₁-C₁₂ substituted or unsubstituted alkyl group, C₆-C₁₄ aryl group, or R₂;
R₂ is a C₁-C₁₂ substituted or unsubstituted alkyl group, or C₆-C₁₄ aryl group;
X is independently oxygen or sulfur;
A₁ is a trialkylsilyl, alkylsulfonate, or C₁-C₁₂ substituted or unsubstituted alkyl group terminating in a nitrile group, with the proviso that if X is oxygen, then A₁ is not an alkylsulfonate; and
A₂, A₃, and A₄ are a difluorophosphite, trialkylsilyl, alkylsulfonate, or C₁-C₁₂ substituted or unsubstituted alkyl group terminating in a nitrile group, with the proviso that if X is oxygen, then A₃ and A₄ are not an alkylsulfonate nor a C₁-C₁₂ substituted or unsubstituted alkyl group terminating in a nitrile group.

Specific examples of the functionalized cyclic ethers according to the disclosure are listed below. These examples are only an illustration and are not meant to limit the disclosure of claims to follow

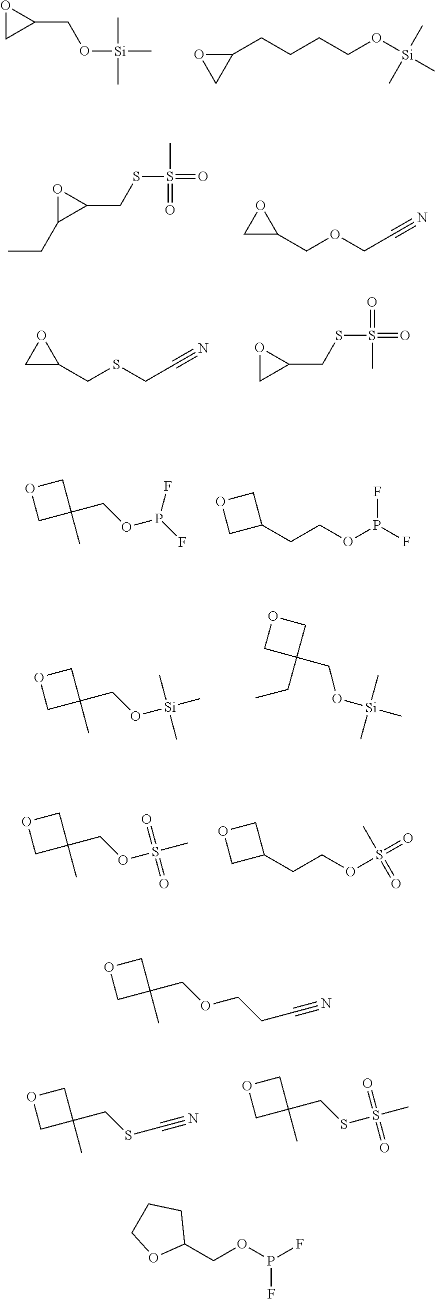

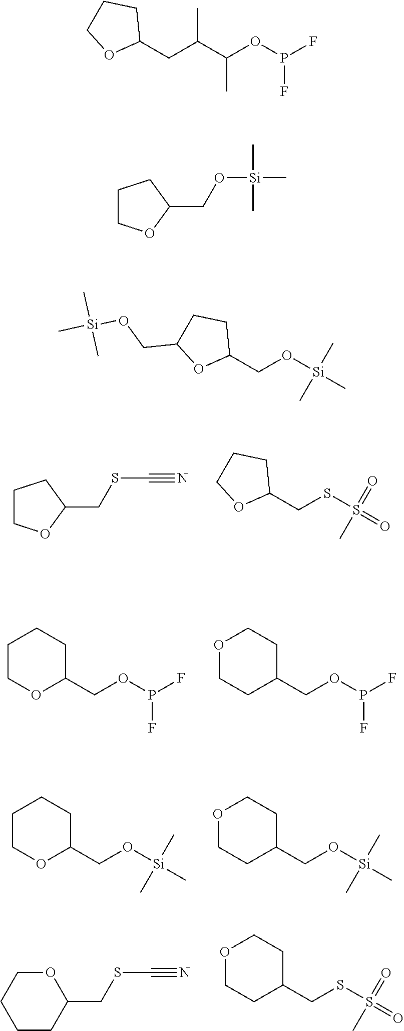

The addition of functionalized cyclic ethers into the Li-ion battery system allows for the polymerization of the cyclic ethers at high temperature, reduction on the anode surface, or oxidation on the surface of the cathode. The resulting polyether film coordinates with the anode or cathode material, which suppresses further reductive or oxidative decomposition of the rest of the electrolyte components that occurs otherwise in contact with the electrode.

A functionalized cyclic ether that comprises a thioether group as part of its molecular structure is also more likely to reduce because sulfur has greater electron withdrawing properties when compared to oxygen. This propensity for reduction results in compounds likely to participate in SEI formation.

The disclosure includes a method for synthesizing the functionalized cyclic ethers, and the use of such molecules in lithium-ion battery electrolytes. These molecules impart greater stability to the electrolytes at higher operating temperatures. In an embodiment of the disclosure, the electrolyte includes a lithium salt in a range of from 10% to 30% by weight. A variety of lithium salts may be used, including, for example, Li(AsF₆); Li(PF₆); Li(CF₃CO₂); Li(C₂F₅CO₂); Li(CF₃SO₃); Li[N(CP₃SO₂)₂]; Li[C(CF₃SO₂)₃]; Li[N(SO₂C₂F₅)₂]; Li(ClO₄); Li(BF₄); Li(PO₂F₂); Li[PF₂(C₂O₄)₂]; Li[PF₄C₂O₄]; lithium alkyl fluorophosphates; Li[B(C₂O₄)₂]; Li[BF₂C₂O₄]; Li₂[B₁₂Z₁₂₋ⱼHⱼ]; Li₂[B₁₀X_{1o-j′}H_{j′}]; or a mixture of any two or more thereof, wherein Z is independent at each occurrence a halogen, j is an integer from 0 to 12 and j′ is an integer from 1 to 10.

In an embodiment of the disclosure, the electrolyte includes an aprotic organic solvent selected from open-chain or cyclic carbonate, carboxylic acid ester, nitrite, ether, sulfone, sulfoxide, ketone, lactone, dioxolane, glyme, crown ether, siloxane, phosphoric acid ester, phosphite, mono- or polyphosphazene or mixtures thereof in a range of from 60% to 90% by weight.

Examples of aprotic organic solvents for generating electrolytes include but are not limited to dimethyl carbonate, ethyl methyl carbonate, diethyl carbonate, methyl propyl carbonate, ethyl propyl carbonate, dipropyl carbonate, propylene carbonate, ethylene carbonate, fluoroethylene carbonate, bis(trifluoroethyl) carbonate, bis(pentafluoropropyl) carbonate, trifluoroethyl methyl carbonate, pentafluoroethyl methyl carbonate, heptafluoropropyl methyl carbonate, perfluorobutyl methyl carbonate, trifluoroethyl ethyl carbonate, pentafluoroethyl ethyl carbonate, heptafluoropropyl ethyl carbonate, perfluorobutyl ethyl carbonate, etc., fluorinated oligomers, methyl propionate, ethyl propionate, butyl propionate, dimethoxyethane, triglyme, dimethylvinylene carbonate, tetraethyleneglycol, dimethyl ether, polyethylene glycols, triphenyl phosphate, tributyl phosphate, hexafluorocyclotriphosphazene, 2-Ethoxy-2,4,4,6,6-pentafluoro-1,3,5,2-5,4-5,6-5 triazatriphosphinine, triphenyl phosphite, sulfolane, dimethyl sulfoxide, ethyl methyl sulfone, ethylvinyl sulfone, allyl methyl sulfone, divinyl sulfone, fluorophenylmethyl sulfone and gamma-butyrolactone.

In an embodiment, the electrolytes include at least one additive to protect the electrodes and electrolyte from degradation. Thus, electrolytes of the present technology may include an additive that is reduced or polymerized on the surface of an electrode to form a passivation film on the surface of an electrode.

In an embodiment, the additive is a substituted or unsubstituted linear, branched, or cyclic hydrocarbon including at least one oxygen atom and at least one aryl, alkenyl or alkynyl group. The passivating film formed from such additives may also be formed from a substituted aryl compound or a substituted or unsubstituted heteroaryl compound where the additive includes at least one oxygen atom.

Representative additives include glyoxal bis(diallyl acetal), tetra(ethylene glycol) divinyl ether, 1,3,5-triallyl-1,3,5-triazine-2,4,6(1H,3H,5H)-trione, 1,3,5,7-tetravinyl-1,3,5,7-tetramethylcyclotetrasiloxane, 2,4,6-triallyloxy-1,3,5-triazine, 1,3,5-triacryloylhexahydro-1,3,5-triazine, 1,2-divinyl furoate, 1,3-butadiene carbonate, 1-vinylazetidin-2-one, 1-vinylaziridin-2-one, 1-vinylpiperidin-2-one, 1 vinylpyrrolidin-2-one, 2,4-divinyl-1,3-dioxane, 2-amino-3-vinylcyclohexanone, 2-amino-3-vinylcyclopropanone, 2 amino-4-vinylcyclobutanone, 2-amino-5-vinylcyclopentanone, 2-aryloxy-cyclopropanone, 2-vinyl-[1,2]oxazetidine, 2 vinylaminocyclohexanol, 2-vinylaminocyclopropanone, 2-vinyloxetane, 2-vinyloxy-cyclopropanone, 3-(N-vinylamino)cyclohexanone, 3,5-divinyl furoate, 3-vinylazetidin-2-one, 3 vinylaziridin-2-one, 3-vinylcyclobutanone, 3-vinylcyclopentanone, 3-vinyloxaziridine, 3-vinyloxetane, 3-vinylpyrrolidin-2-one, 2-vinyl-1,3-dioxolane, acrolein diethyl acetal, acrolein dimethyl acetal, 4,4-divinyl-3-dioxolan-2-one, 4-vinyltetrahydropyran, 5-vinylpiperidin-3-one, allylglycidyl ether, butadiene monoxide, butyl-vinyl-ether, dihydropyran-3-one, divinyl butyl carbonate, divinyl carbonate, divinyl crotonate, divinyl ether, divinyl ethylene carbonate, divinyl ethylene silicate, 1,3 propane sultone, 1,3 propene sultone, divinyl ethylene sulfate, divinyl ethylene sulfite, divinyl methoxypyrazine, divinyl methylphosphate, divinyl propylene carbonate, ethyl phosphate, methoxy-o-terphenyl, methyl phosphate, oxetan-2-yl-vinylamine, oxiranylvinylamine, vinyl carbonate, vinyl crotonate, vinyl cyclopentanone, vinyl ethyl-2-furoate, vinyl ethylene carbonate, 4-fluoro-1,3-dioxolan-2-one, vinyl ethylene silicate, vinyl ethylene sulfate, vinyl ethylene sulfite, vinyl methacrylate, vinyl phosphate, vinyl-2-furoate, vinylcylopropanone, vinylethylene oxide, β-vinyl-γ-butyrolactone or a mixture of any two or more thereof. In some embodiments, the additive may be a cyclotriphosphazene that is substituted with F, alkyloxy, alkenyloxy, aryloxy, methoxy, allyloxy groups or combinations thereof. For example, the additive may be a (divinyl)-(methoxy)(trifluoro)cyclotriphosphazene, (trivinyl)(difluoro)(methoxy)cyclotriphosphazene, (vinyl)(methoxy)(tetrafluoro)cyclotriphosphazene, (aryloxy)(tetrafluoro)(methoxy)cyclotriphosphazene or (diaryloxy)(trifluoro)(methoxy)cyclotriphosphazene compounds or a mixture of two or more such compounds.

In some embodiments the additive is a sulfur-containing compound, phosphorus-containing compound, boron-containing compound, silicon-containing compound, fluorine-containing compound, nitrogen-containing compound, compound containing at least one unsaturated carbon-carbon bond, carboxylic acid anhydride or the mixtures thereof. In some embodiments, the additive is vinyl carbonate, vinyl ethylene carbonate, or a mixture of any two or more such compounds.

In some embodiments the additive is a fully or partially halogenated phosphoric acid ester compound, an ionic liquid, or mixtures thereof. The halogenated phosphoric acid ester may include 4-fluorophenyldiphenylphosphate, 3,5-difluorophenyldiphenylphosphate, 4-chlorophenyldiphenylphosphate, trifluorophenylphosphate, heptafluorobutyldiphenylphosphate, trifluoroethyldiphenylphosphate, bis(trifluoroethyl)phenylphosphate, and phenylbis(trifluoroethyl)phosphate. The ionic liquids may include tris(N-ethyl-N-methylpyrrolidinium)thiophosphate bis(trifluoromethylsulfonyl)imide, tris(N-ethyl-N-methylpyrrolidinium) phosphate bis(trifluoromethylsulfonyl)imide, tris(N-ethyl-N-methylpiperidinium)thiophosphate bis(trifluoromethylsulfonyl)imide, tris(N-ethyl-N-methylpiperidinium)phosphate bis(trifluoromethylsulfonyl)imide, N-methyl-trimethylsilylpyrrolidinium bis(trifluoromethylsulfonyl)imide, N-methyl-trimethylsilylpyrrolidinium hexafluorophosphate. The additives discussed above are present in a range of from 0.01% to 10% by weight.

In another embodiment, an electrochemical energy storage device is provided that includes a cathode, an anode and an electrolyte including an ionic liquid as described herein. In one embodiment, the electrochemical energy storage device is a lithium secondary battery. In some embodiments, the secondary battery is a lithium battery, a lithium-ion battery, a lithium-sulfur battery, a lithium-air battery, a sodium ion battery, or a magnesium battery. In some embodiments, the electrochemical energy storage device is an electrochemical cell, such as a capacitor. In some embodiments, the capacitor is an asymmetric capacitor or supercapacitor. In some embodiments, the electrochemical cell is a primary cell. In some embodiments, the primary cell is a lithium/MnO₂ battery or Li/poly(carbon monofluoride) battery. In some embodiments, the electrochemical energy storage device is a solar cell.

In an embodiment, a secondary battery is provided including a positive and a negative electrode separated from each other using a porous separator and the electrolyte described herein.

Suitable cathode materials for a secondary battery including the electrolyte described herein include those such as, but not limited to, vanadium oxide, lithium peroxide, sulfur, polysulfide, a lithium carbon monofluoride (also known as LiCFₓ) or mixtures of any two or more thereof, carbon-coated olivine cathodes such as LiFePO₄, and lithium metal oxides such as LiCoO₂, LiNiO₂, LiNiₓCo_{y}Met_{z}O₂, LiMn_{0.5}Ni_{0.5}O₂, LiMn_{0.1}Co_{0.1}Ni_{0.8}O₂, LiMn_{0.2}Co_{0.2}Ni_{0.6}O₂, LiMn_{0.3}Co_{0.2}Ni_{0.5}O₂, LiMn_{0.33}Co_{0.33}Ni_{0.33}O₂, LiMn₂O₄, LiFeO₂, Li_{1+x′}Ni_{α}Mn_{β}Co_{y}Met′_{δ}O_{2-z′}F_{z′}, or A_{n′}B₂(XO₄)₃, wherein Met is Al, Mg, Ti, B, Ga, Si, Mn or Co; Met′ is Mg, Zn, Al, Ga, B, Zr or Ti; A is Li, Ag, Cu, Na, Mn, Fe, Co, Ni, Cu or Zn; B is Ti, V, Cr, Fe or Zr; X is P, S, Si, W or Mo; and wherein 0≤x≤0.3, 0≤y≤0.5, 0≤z≤0.5, 0≤x′≤0.4, 0≤α≤1, 0≤β≤1, 0≤γ≤1, 0≤δ≤0.4, 0≤z′≤0.4 and 0≤n′≤3. In other embodiments, an olivine cathode has a formula of Li₁₊ₓFe_{1z}Met″_{y}PO₄₋ₘX′ₙ, wherein Met″ is Al, Mg, Ti, B, Ga, Si, Ni, Mn or Co; X′ is S or F; and wherein 0≤x≤0.3, 0≤y≤0.5, 0≤z≤0.5, 0≤m≤0.5 and 0≤n≤0.5.

Suitable anodes include those such as lithium metal, graphitic materials, amorphous carbon, carbon nanotubes, Li₄Ti₅O₁₂, tin alloys, silicon, silicon alloys, intermetallic compounds, or mixtures of any two or more such materials. Suitable graphitic materials include natural graphite, artificial graphite, graphitized meso-carbon microbeads (MCMB) and graphite fibers, as well as any amorphous carbon materials. In some embodiments, the anode and cathode electrodes are separated from each other by a porous separator.

In some embodiments, the anode is a composite anode including active materials such as silicon and silicon alloys, and a conductive polymer coating around the active material. The active material may be in the form of silicon particles having a particle size of between about 1 nm and about 100 μm. Other suitable active materials include but are not limited to hard-carbon, graphite, tin, and germanium particles. The polymer coating material can be cyclized using heat treatment at temperatures of from 200° C. to 400° C. to thereby convert the polymer to a ladder compound by crosslinking polymer chains. Specific polymers that can be used include but are not limited to polyacrylonitrile (PAN) where the cyclization changes the nitrile bond (C≡N) to a double bond (C═N). The polymer material forms elastic but robust films to allow for controlled fragmentation/pulverization of silicon particles within the polymer matrix.

Additionally, the PAN matrix also provides a path for Li-ion mobility thus enhancing the conductivity of the composite anode. The resultant anode material can overcome expansion and conductivity challenges of silicon-based anodes, such as by providing binders that can prevent expansion of silicon particles and conductive additives to provide a path for Li-ion mobility. In some embodiments, the polymer is about 10 wt. % to 40 wt. % of the anode composite material. Additional description of these Si-PAN composite anodes is provided in U.S. Pat. Nos. 10,573,884 and 10,707,481, both of which are hereby incorporated by reference in their entirety.

The separator for the lithium battery may be a microporous polymer film. Examples of polymers for forming films include polypropylene, polyethylene, nylon, cellulose, nitrocellulose, polysulfone, polyacrylonitrile, polyvinylidene fluoride, polybutene, or copolymers or blends of any two or more such polymers. In some instances, the separator is an electron beam-treated micro-porous polyolefin separator. The electron treatment can increase the deformation temperature of the separator and can accordingly enhance thermal stability at high temperatures. Additionally, or alternatively, the separator can be a shut-down separator. The shut-down separator can have a trigger temperature above about 130° C. to permit the electrochemical cells to operate at temperatures up to about 130° C.

The disclosure will be further illustrated with reference to the following specific examples. It is understood that these examples are given by way of illustration and are not meant to limit the disclosure or the claims to follow.

### Example 1—Synthesis of Glycidyl Trimethylsilyl Ether

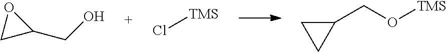 [SMILES: OCC1CO1 ; C[Si](C)(C)Cl ; C[Si](C)(C)OCC1CC1]

1. To a 100 mL 3-neck flask equipped with a magnetic stirring bar, water-cooled condenser, N₂ inlet and thermocouple were added glycidol and DCM (40 mL). The flask was placed in an ice bath.
2. While stirring at 5° C., triethylamine was added by pipet and an exotherm to 16° C. was observed.
3. While stirring at 5° C., TMS-Cl was slowly added by syringe over 20 min. and an exotherm under 12° C. was maintained. A white solid ppt (presumably triethylamine-HCl) quickly formed and the mixture gradually returned to RT as the ice bath warmed up. The mixture stirred at RT for 4 h.
4. DI water (2×30 mL) was added, and the organic phase was extracted into DCM, separated, dried over MgSO₄, filtered and the solvent stripped by rotary evaporation to oil. The oil was pumped under high vacuum and passed through a 0.45 μm GMF filter.

Yield: pale yellow oil, 11.6 g (98%).

H+ NMR: (CDC₁₃) δ ppm 3.82 (dd, 1H), 3.60 (dd, 1H), 3.09 (m, 1H), 2.78 (t, 1H), 2.62 (t, 1H), 0.14 (s, 9H).

### Example 2—Synthesis of 2-((Trimethylsiloxy)methyl)tetrahydrofuran

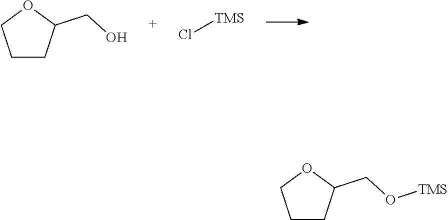 [SMILES: OCC1CCCO1 ; C[Si](C)(C)Cl ; C[Si](C)(C)OCC1CCCO1]

1. To a 100 mL 3-neck flask equipped with a magnetic stirring bar, water-cooled condenser, N₂ inlet and thermocouple were added tetrahydrofurfuryl alcohol and DCM (40 mL).
2. While stirring at RT, triethylamine was added by pipet and an exotherm to 29° C. was observed.
3. While stirring at RT, TMS-Cl was slowly added by syringe over 20 min. and an exotherm under 45° C. was maintained. A white solid ppt (presumably triethylamine-HCl) quickly formed and the mixture gradually returned to RT. The mixture stirred at RT for 2 h.
4. DI water (2×30 mL) was added, and the organic phase was extracted into DCM, separated, dried over MgSO₄, filtered and the solvent stripped by rotary evaporation to oil. The oil was pumped under high vacuum and passed through a 0.45 μm GMF filter. Yield: pale yellow oil, 5.7 g (84%).

H+ NMR: (CDC₁₃) δ ppm 3.86 (m, 1H), 3.75 (m, 1H), 3.68 (m, 1H), 3.56 (d, 2H), 1.87 (m, 3H), 1.64 (m, 1H), 0.12 (s, 9H).

### Example 3—Synthesis of (3-methyloxetan-3-yl)methyl methanesulfonate

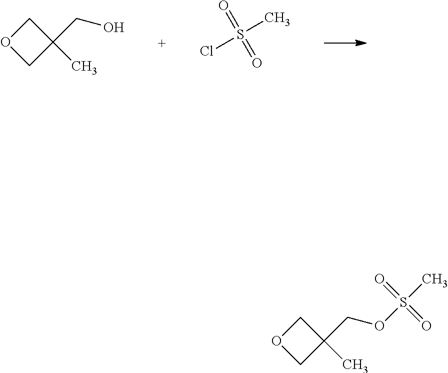

1. To a 100 mL 3-neck flask equipped with a magnetic stirring bar, water-cooled condenser, N₂ inlet and thermocouple were added 3-methyl-3-oxetane methanol and DCM (40 mL).
2. While stirring at RT, triethylamine was added by pipet and an exotherm to 29° C. was observed. The flask was placed in an ice bath.
3. While stirring at 8° C., methanesulfonyl chloride was slowly added by syringe over 10 min. and an exotherm under 18° C. was maintained. A white solid ppt (presumably triethylamine-HCl) quickly formed and the mixture gradually returned to RT. The mixture stirred at RT for 2 h.
4. DI water (30 mL) was added, and the organic phase was extracted into DCM, separated, dried over MgSO₄, filtered and the solvent stripped by rotary evaporation to oil. The oil was pumped under high vacuum and passed through a 0.45 μm GMF filter. Yield: pale yellow oil, 5.1 g (96%).

H+ NMR: (CDC₁₃) δ ppm 4.51 (d, 2H), 4.42 (d, 2H), 4.32 (s, 2H), 3.07 (s, 3H), 1.39 (s, 3H).

### Example 4—Synthesis of 3-methyl-3-oxetanylmethyldifluoro-phosphite

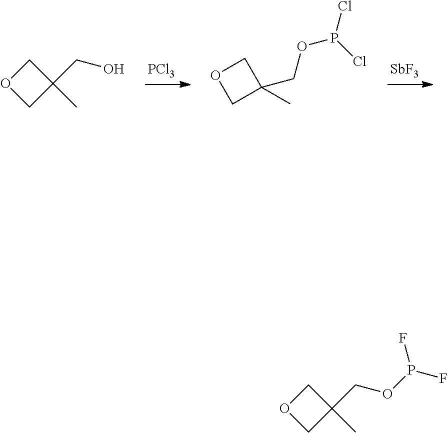

### Step A: Synthesis of 3-methyl-3-oxetanylmethyldichloro-phosphite

1. To a 100 mL 3-neck half-jacketed flask equipped with a magnetic stirring bar, nitrogen (N₂) inlet, addition funnel and thermocouple were added 60 mL of dichloromethane (DCM) and phosphoroustrichloride. The flask was cooled to 0° C.
2. While stirring at room temperature (RT), a solution of 3-methyl-3-oxetane methanol in DCM (20 mL) was treated with triethylamine dropwise by pipet. No exotherm was observed. 3. While stirring at 0° C., the 3-methyl-3-oxetanyl reagent was slowly added dropwise by addition funnel over a 2 h period (˜1 drop/sec) and an exotherm under 2.0° C. was maintained. The mixture slowly turned cloudy, and a salt byproduct (presumably triethylamine-HCl) was formed.
4. After addition was complete, some solid was collected from the cold mixture by vacuum filtration. The solvent was stripped by rotary evaporation. An oily solid precipitate (ppt) formed and was slurried in hexane (60 mL). The solid was removed by vacuum filtration and washed with hexane (3×10 mL) and the filtrate was used as-is assuming theoretical yield, 7.9 g.

### Step B: Synthesis of 3-methyl-3-oxetanylmethyldifluoro-phosphite

5. To a 250 mL round bottom (RB) flask containing the filtrate of the Step A product in hexane (80 mL) was added a magnetic stirring bar and thermocouple.
6. While stirring at RT, antimony(III)fluoride was slowly added as a solid. An exotherm to 29.0° C. was observed. A dense pale white oily residue was quickly deposited on the bottom (presumably SbC₁₃).
7. The mixture stirred for 2 h. The solvent was decanted from the residue and passed through a 0.45 μm GMF filter unit and stripped by rotary evaporation. Yield: clear yellow oil, 4.8 g (73%).
8. Distillation: pot: 90-100° C.; vapor: 52-67° C.; p: 0.3-0.25 mmHg. Yield: colorless oil, 6.4 g.

H+ NMR: δ ppm 4.41-4.37 (q, 2H), 3.91-3.89 (t, 2H), 3.79 (s, 2H), 0.91 (s, 3H).

F19 NMR: (CDC₁₃) δ ppm −64.9 (s, 1F), −67.44 (s, 1F).

P31 NMR: (CDC₁₃) δ ppm 115.43 (s), 109.57 (s).

### Example 5—Synthesis of (oxan-2-yl)methyl phosphorodifluoridoite

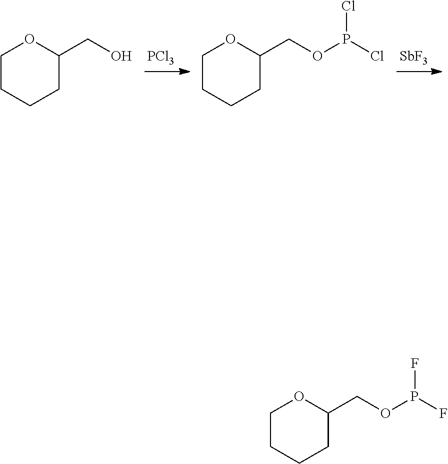

### Step A: Synthesis of tetrahydropyranyl-methyl-dichloro-phosphite

1. To a 100 mL 3-neck half-jacketed flask equipped with a magnetic stirring bar, N₂ inlet, addition funnel and thermocouple were added ethyl acetate (60 mL) and phosphoroustrichloride. The flask was cooled to −5° C.
2. While stirring at RT, a solution of tetrahydropyran-2-methanol in ethyl acetate (30 mL) was treated with 4-dimethylamino pyridine dropwise by pipet. An exotherm to 25° C. was observed.
3. While stirring at −5° C., the tetrahydropyranylmethyl reagent was slowly added dropwise by addition funnel over a 1 h period (˜1 drop/sec) and an exotherm under 2.0° C. was maintained. The mixture quickly turned cloudy, and a salt byproduct (presumably DMAP-HCl) was formed.
4. The byproduct was removed by vacuum filtration and washed with ethyl acetate (3×10 mL) and the filtrate was passed through a 0.45 μm GMF filter unit and stripped by rotary evaporation, 7.5 g.

### Step B: Synthesis of tetrahydropyranyl-methyl-difluoro-phosphite

5. To the 250 mL RB flask containing the filtrate of the Step A product in hexane (150 mL) was added a magnetic stirring bar and thermocouple.
6. While stirring at RT, antimony(III)fluoride was slowly added as a solid. An exotherm to 32.0° C. was observed. A dense white oily residue was quickly deposited on the bottom (presumably SbC₁₃). The mixture slowly returned to RT and stirred for 3 h.
7. The reaction solvent was decanted from the residue and passed through a 0.45 μm GMF filter unit and stripped by rotary evaporation. Yield: clear colorless oil, 3.3 g (52%).

H+ NMR: (CDC₁₃) δ ppm 4.04 (m, 2H), 3.47 (m, 2H), 1.90 (m, 1H), 1.55 (m, 4H), 1.34 (m, 2H).

F19 NMR: (CDC₁₃) δ ppm −47.25 (dd, 1F), −49.95 (dd, 1F).

P31 NMR: (CDC₁₃) δ ppm 119.57 (s), 113.17 (s), 106.78 (s).

### Example 6—Synthesis of (oxolan-2-yl)methyl phosphorodifluoridoite

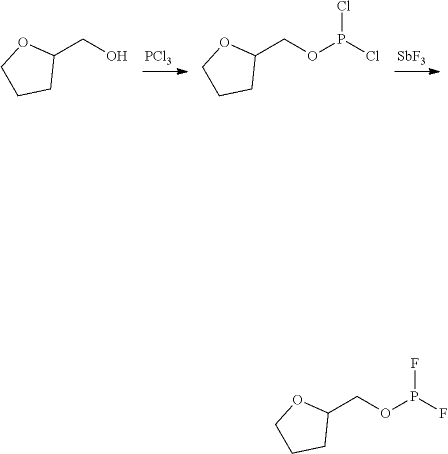

### Step A: Synthesis of tetrahydrofurfuryldichloro-phosphite

1. To a 100 mL 3-neck half-jacketed flask equipped with a magnetic stirring bar, N2 inlet, addition funnel and thermocouple were added ethyl acetate (60 mL) and phosphoroustrichloride. The flask was cooled to −5° C.
2. While stirring at RT, a solution of tetrahydrofurfuryl alcohol in ethyl acetate (30 mL) was treated with dimethylamino pyridine dropwise by pipet. No exotherm was observed.
3. While stirring at −5° C., the 3-methyl-3-oxetanyl reagent was slowly added dropwise by addition funnel over a 2 h period (1 drop/sec) and an exotherm under 2.0° C. was maintained. The mixture slowly turned cloudy, and a salt byproduct (presumably DMAP-HCl) was formed.
4. The byproduct was removed by vacuum filtration and washed with ethyl acetate (3×10 mL) and the filtrate was passed through a 0.45 μm GMF filter unit and stripped by rotary evaporation, 7.8 g.

### Step B: Synthesis of (oxolan-2-yl)methyl phosphorodifluoridoite

5. To the 250 mL RB flask containing the filtrate of the Step A product in hexane (80 mL) was added a magnetic stirring bar and thermocouple.
6. While stirring at RT, antimony(III)fluoride was slowly added as a solid. An exotherm to 32.0° C. was observed. A dense white oily residue was quickly deposited on the bottom (presumably SbCl3). The mixture slowly returned to RT and stirred for 3 h.
7. The reaction solvent was decanted from the residue and passed through a 0.45 μm GMF filter unit and stripped by rotary evaporation. Yield: clear colorless oil, 2.4 g (36%).

H+ NMR: (CDCl3) δ ppm 4.16-4.04 (m, 3H), 4.00 (m, 1H), 3.88 (m, 1H), 3.83 (m, 1H), 2.02-1.92 (m, 3H), 1.67 (m, 1H).

F19 NMR: (CDCl3) δ ppm −79.69 (t, 1F), −81.77 (t, 1F).

P31 NMR: (CDCl3) δ ppm 9.62, 9.19, 8.68 (t, 1P).

### Example 7—Synthesis of glycidyl cyanomethyl ether (GCME)

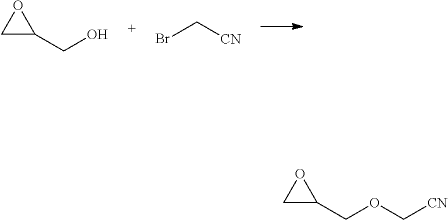

1. To a 100 mL 3-neck flask equipped with a magnetic stirring bar, water-cooled condenser, N2 inlet and thermocouple was added potassium tert-butoxide and DCM (30 mL). The flask was placed in an ice bath.
2. While stirring at 3° C., glycidol was added dropwise to the white slurry. A mild exotherm to 10° C. was observed and the mixture became partially clear and continued to stir for 30 min.
3. While stirring at 3° C., bromoacetonitrile was added dropwise by syringe. An exotherm to under 10° C. was maintained and the mixture quickly turned dark brown. A white solid ppt (presumably, KBr) was quickly formed and the mixture slowly returned to RT and stirred for 2 h.
4. DI water (100 mL) was added, and the mixture was poured into a separatory funnel. The upper brown water layer was separated from the lower yellowish DCM layer. The upper layer was extracted into DCM (2×30 mL), separated, combined with the prior DCM layer, dried over MgSO₄ and the solvent stripped by rotary evaporation. Yield: light yellow oil, 6.3 g (83%).
5. The oil was pumped under high vacuum. Yield: light yellow oil, 5.1 g (67%).

H+ NMR: (CDCl3) δ ppm 4.35 (d, 2H), 3.97 (dd, 1H), 3.48 (q, 1H), 3.19 (m, 1H), 2.84 (t, 1H), 2.65 (q, 1H).

### Electrolytes for NMC811/Graphite Cells

Electrolyte compositions were prepared in a dry argon filled glovebox by combining all the electrolyte components in a glass vial and stirring for 24 hours to ensure complete dissolution of the salts. A functionalized cyclic ether is added to a base electrolyte composition comprising a 3:7 by weight mixture of ethylene carbonate, EC, and ethyl methyl carbonate, EMC, and 1 M lithium hexafluorophosphate, LiPF₆, as a Li⁺ ion conducting salt, dissolved therein. The conventional additive vinylene carbonate(VC), was added to the base formulation at 2 weight percent. The Embodiment Examples (EE1-6) use representative Example molecules (Examples 1-6) at a concentration of 1 weight percent. All example molecules were readily miscible in the base formulation. The electrolyte components and functionalized cyclic ether used are summarized in Table A.

TABLE A
Electrolyte Compositions for Li-ion cells

| | | Functionalized Cyclic Ether |
|---|---|---|
| Electrolyte | Base Formulation | Weight (%) |
| Comparative | 1.0M LiPF₆ in | None |
| Example 1 | EC:EMC (3:7) | |
| (CE1) | VC: 2% | |
| Embodiment | 1.0M LiPF₆ in | Glycidyl trimethylsilyl ether: 1% |
| Example 1 | EC:EMC (3:7) | |
| (EE1) | VC: 2% | |
| Embodiment | 1.0M LiPF₆ in | 2-((Trimethylsiloxy)methyl)tetra- |
| Example 2 | EC:EMC (3:7) | hydrofuran: 1% |
| (EE2) | VC: 2%, | |
| Embodiment | 1.0M LiPF₆ in | (3-methyloxetan-3-yl)methyl |
| Example 3 | EC:EMC (3:7) | methanesulfonate: 1% |
| (EE3) | VC: 2% | |
| Embodiment | 1.0M LiPF₆ in | (3-methyloxetan-3-yl)methyl |
| Example 4 | EC:EMC (3:7) | phosphorodifluoridoite: 1% |
| (EE4) | VC: 2% | |
| Embodiment | 1.0M LiPF₆ in | (oxan-2-yl)methyl phosphorodi- |
| Example 5 | EC:EMC (3:7) | fluoridoite: 1% |
| (EE5) | VC: 2% | |
| Embodiment | 1.0M LiPF₆ in | (oxolan-2-yl)methyl |
| Example 6 | EC:EMC (3:7) | phosphorodifluoridoite: 1% |
| (EE6) | VC: 2% | |

### NMC811/Graphite Cell Data

The electrolyte compositions prepared are used as electrolytes in 230 mAh Li-ion pouch cells comprising lithium nickel manganese cobalt oxide (NMC811) cathode active material and artificial graphite as the anode active material. A conventional polyethylene film is used as a separator. In each cell, 0.9 mL of electrolyte composition was added and allowed to soak in the cell for 1 hour. The cells are vacuum sealed, and primary charged before wetting at 25° C. for 10 hours. The cells were then charged to 3.7 V at C/10 rate at 60° C. before degassing, followed by vacuum sealing. After degassing, the cells were charged and discharged twice between 4.2 to 3.0 V at C/10 rate at 25° C. The direct current internal resistance (DCIR) is then collected by applying a 10 second 1C discharge pulse to the cell and measuring the voltage drop of the cell. The results are summarized in Table B. With addition of 1 wt. % functionalized cyclic ether, the initial cell data is comparable to reference electrolyte and the nominal capacity of the cells is achieved. However, the first coulombic efficiency can be improved with the addition of the disclosed functionalized cyclic ether. Further, the incorporation of said additives can also reduce the resistance of the cell relative to the comparative example.

TABLE B
Initial Cell Data for NMC811/Graphite cells

| | 1^{st} Coulombic | Formation | DCIR (10 s 1 C |
|---|---|---|---|
| | Efficiency | Discharge Capacity | Discharge) |
| Electrolyte | (%) | (mAh) | (mΩ) |
| CE1 | 86.30 | 240.79 | 475.7 |
| EE1 | 86.77 | 242.63 | 472.7 |
| EE2 | 86.12 | 241.33 | 439.2 |
| EE3 | 86.91 | 242.91 | 450.9 |
| EE4 | 85.60 | 240.61 | 484.2 |
| EE5 | 86.50 | 240.32 | 499.3 |
| EE6 | 86.32 | 239.91 | 605.4 |

The cells are then subjected to a high temperature storage test after the initial formation cycling. The cells are charged to 100% state-of-charge at 4.2 V at a 0.3° C. rate and then the thickness of the cell is measured at the center of the active stack. The cells are then stored in a 60° C. environmental chamber for two weeks. After two weeks, the change in thickness and capacity of the cells was measured. The results are summarized in Table C. As can be seen in Table C, all cells demonstrated some swelling attributable to gas generation and loss of capacity. However, the incorporation of the functionalized cyclic ether additive results in significantly reduced gas generation relative to the comparative example. Gas generation at elevated temperature is largely a function of the accelerated decomposition of the cathode material interacting with the electrolyte. The functionalized cyclic ether is thus shown to greatly inhibit these reactions. Further, the capacity retention of the cells can also then be improved with these additives as shown in Table C.

TABLE C
60° C. Storage data for NMC811/Graphite cells

| | Initial Cell | % Thickness | Initial | |
|---|---|---|---|---|
| | thickness | growth at 2 | Capacity | % Capacity at |
| Electrolyte | (mm) | weeks | (mAh) | 2 weeks |
| CE1 | 4.10 | 5.5 | 231.28 | 90.2 |
| EE1 | 4.03 | 5.0 | 230.41 | 88.1 |
| EE2 | 4.10 | 4.5 | 229.32 | 89.8 |
| EE3 | 4.10 | 2.1 | 231.91 | 91.4 |
| EE4 | 4.10 | 3.0 | 230.67 | 90.6 |
| EE5 | 4.07 | 2.1 | 227.09 | 91.9 |
| EE6 | 4.06 | 1.8 | 227.67 | 92.2 |

### Electrolytes for LCO/Graphite Cells

Electrolyte compositions were prepared in a dry argon filled glovebox by combining all the electrolyte components in a glass vial and stirring for 24 hours to ensure a completely homogeneous mixture. The individual components of the electrolyte compositions are ethylene carbonate (EC), propylene carbonate (PC), ethyl propionate (EP), propyl propionate (PP), fluoroethylene carbonate (FEC), 1,3-propanesultone (PaS), 1,3-propenesultone (PeS), 1,3,6-hexacarbonitrile (HTCN), lithium hexafluorophosphate (LiPF6), and lithium difluoro(oxalato)borate (LiDFOB). The base formulation for all formulations tested in LCO/Graphite cells was 1M LiPF6 in EC/PC/EP/PP 20/10/25/45 weight basis solvent, with 0.7 wt. % LiDFOB, 4% PaS, 7% FEC, and 3% HTCN. The embodiment examples use the representative example molecule glycidyl cyanomethyl ether (GCME) as per the present disclosure at a concentration of 0.5 weight percent. GCME is readily miscible in the solution. The electrolyte components and functionalized cyclic ether used are summarized in Table D.

TABLE D
Electrolyte compositions for Li-ion cells

| | Functionalized Cyclic |
|---|---|
| Electrolyte | Ether Weight (%) |
| Comparative Example 2 (CE2) | None |
| Embodiment Example 7 (EE7) | 0.5 wt % GCME |

### LCO/Graphite Cell Data.

The electrolyte compositions prepared are used as electrolytes in 230 mAh Li-ion pouch cells comprising lithium cobalt oxide (LCO) cathode active material and artificial graphite as the anode active material. A conventional polyethylene film is used as a separator. In each cell, 0.9 mL of electrolyte composition was added and allowed to soak in the cell for 1 hour. The cells are vacuum sealed, and primary charged before wetting at 25° C. for 10 hours. The cells were then charged to 3.8 V at C/10 rate at 25° C. before degassing, followed by vacuum sealing. After degassing, the cells were charged and discharged twice between 4.45 to 3.0 V at C/10 rate at 25° C. The direct current internal resistance (DCIR) is then collected by applying a 10 second 1C discharge pulse to the cell and measuring the voltage drop of the cell, this is done at 50% state-of-charge (SOC). The results are summarized in Table E. With addition of 0.5 wt. % GCME, the initial cell data is comparable to reference electrolyte and the nominal capacity of the cells is achieved. However, it is observed that the incorporation of GCME reduces the resistance of the cell relative to the comparative example.

TABLE E
Initial Cell Data for LCO/Graphite cells

| | 1^{st} Coulombic | Formation | DCIR (10 s 1 C |
|---|---|---|---|
| | Efficiency | Discharge Capacity | Discharge) |
| Electrolyte | (%) | (mAh) | (mΩ) |
| CE2 | 91.06 | 239.51 | 690.9 |
| EE7 | 92.49 | 238.77 | 650.9 |

After the activation and formation procedure the cells are subjected to a high temperature storage test. The cells are charged to 100% SOC at 4.45 V at a 0.3C rate and then the thickness of the cell is measured at the center of the active stack. The alternating current internal resistance (AC-IR) is measured at 100% SOC with a Hioki battery impedance meter. The cells are then stored in a 60° C. environmental chamber for three weeks. After three weeks, the change in thickness and resistance was measured. The results are summarized in Table F. As can be seen in Table F, all cells demonstrated some swelling attributable to gas generation and increase in resistance. Gas generation at elevated temperature is largely a function of the accelerated decomposition of the cathode material interacting with the electrolyte, also resulting in increased resistance and lower capacity. However, the incorporation of the embodiment compound, GCME, results in significantly reduced gas generation and resistance growth relative to the comparative example. GCME is thus shown to greatly inhibit these reactions.

TABLE F
60° C. Storage data for LCO/Graphite cells

| | Initial Cell | % Thickness | | |
|---|---|---|---|---|
| | thickness | growth at 3 | Initial Cell | % IR growth |
| Electrolyte | (mm) | weeks | AC-IR (mΩ) | at 3 weeks |
| CE2 | 4.16 | 2.6 | 113 | 140.8 |
| EE7 | 4.07 | 1.9 | 110 | 132.4 |

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follows.

What is claimed is:

1. An electrolyte composition comprising:
an aprotic organic solvent system;
a metal salt; and
at least one functionalized cyclic ether according to formulas I, II, III or IV: 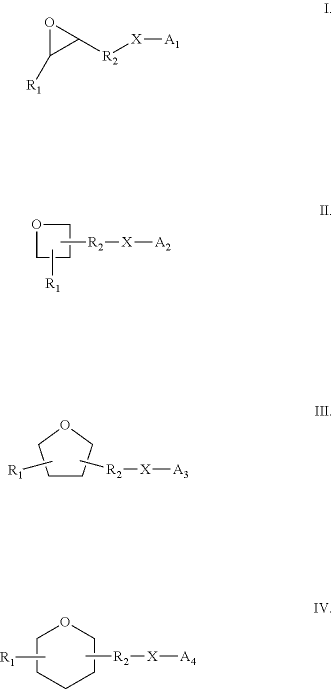
wherein:
R₁ is a hydrogen, halogen, C₁-C₁₂ substituted or unsubstituted alkyl group, C₆-C₁₄ aryl group, or R₂;
R₂ is a C₁-C₁₂ substituted or unsubstituted alkyl group, or C₆-C₁₄ aryl group;
X is independently oxygen or sulfur;
A₁ is a trialkylsilyl, alkylsulfonate, C₁-C₁₂ substituted or unsubstituted alkyl group terminating in a nitrile group, with the proviso that if X is oxygen, then A₁ is not an alkylsulfonate; and
A₂, A₃, and A₄ are a difluorophosphite, trialkylsilyl, alkylsulfonate, or a C₁-C₁₂ substituted or unsubstituted alkyl group terminating in a nitrile group, with the proviso that if X is oxygen, then A₃ and A₄ are not an alkylsulfonate nor a C₁-C₁₂ substituted or unsubstituted alkyl group terminating in a nitrile group.

2. The electrolyte composition of claim 1, wherein the functionalized cyclic ether comprises (3-methyloxetan-3-yl)methyl phosphorodifluoridoite, (oxolan-2-yl)methyl phosphorodifluoridoite, (oxan-2-yl)methyl phosphorodifluoridoite, glycidyl cyanomethyl ether, 3-[(3-methyloxetan-3-yl)methoxy]propanenitrile, glycidyl trimethylsilyl ether, 3-methyl-3-[[(trimethylsilyl)oxy]methyl]oxetane, 2-((trimethylsiloxy)methyl)tetrahydrofuran, trimethyl[(tetrahydropyran-2-yl)methoxy]-silane, and (3-methyloxetan-3-yl)methyl methanesulfonate.

3. The electrolyte composition of claim 1, wherein the functionalized cyclic ether is present in a concentration of from 0.001 wt. % to 25 wt. % in the electrolyte composition.

4. The electrolyte composition of claim 1, wherein the aprotic organic solvent system comprises an open-chain or cyclic carbonate, carboxylic acid ester, nitrite, ether, sulfone, ketone, lactone, dioxolane, glyme, crown ether, siloxane, phosphoric acid ester, phosphite, mono- or polyphosphazene or mixtures thereof.

5. The electrolyte composition of claim 1, wherein the aprotic organic solvent system is present in a concentration of from 60 wt. % to 90 wt. % in the electrolyte composition.

6. The electrolyte composition of claim 1, wherein the cation of the metal salt is an alkali metal.

7. The electrolyte composition of claim 6, wherein the alkali metal is lithium or sodium.

8. The electrolyte composition of claim 1, wherein the cation of the metal salt is aluminum or magnesium.

9. The electrolyte composition of claim 1, wherein the metal salt is present in a concentration of from 10 wt. % to 30 wt. % in the electrolyte composition.

10. The electrolyte composition of claim 1, further comprising at least one additive.

11. The electrolyte composition of claim 10, wherein the additive comprises a sulfur-containing compound, phosphorus-containing compound, boron-containing compound, silicon-containing compound, fluorine-containing compound, nitrogen-containing compound, compound containing at least one unsaturated carbon-carbon bond, carboxylic acid anhydride or the mixtures thereof.

12. The electrolyte composition of claim 10, wherein the additive is present in a concentration of from 0.01 wt. % to 10 wt. % in the electrolyte composition.

13. An electrochemical energy storage device comprising:
a cathode;
an anode;
a separator; and
a formulation comprising the electrolyte composition according to claim 1.

14. The device of claim 13, wherein the cathode comprises a lithium metal oxide, spinel, olivine, carbon-coated olivine, vanadium oxide, lithium peroxide, sulfur, polysulfide, a lithium carbon monofluoride or mixtures of any two or more thereof.

15. The device of claim 14, wherein the lithium metal oxide is, LiCoO₂, LiNiO₂, LiNiₓCo_{y}Met_{z}O₂, LiMn_{0.5}Ni_{0.5}O₂, LiMn_{0.1}Co_{0.1}Ni_{0.8}O₂, LiMn_{0.2}Co_{0.2}Ni_{0.6}O₂, LiMn_{0.3}Co_{0.2}Ni_{0.5}O₂, LiMn_{0.33}Co_{0.33}Ni_{0.33}O₂, LiMn₂O₄, LiFeO₂, Li_{1+x′}Ni_{α}Mn_{β}Co_{γ}Met_{′δ}O_{2-z′}F_{z′}, A_{n′}B₂(XO₄)₃, where Met is Al, Mg, Ti, B, Ga, Si, Mn or Co; Met′ is Mg, Zn, Al, Ga, B, Zr or Ti; A is Li, Ag, Cu, Na, Mn, Fe, Co, Ni, Cu or Zn; B is Ti, V, Cr, Fe or Zr; X is P, S, Si, W or Mo; and wherein 0≤x≤0.3, 0≤y≤0.5, 0≤z≤0.5, 0≤x′≤0.4, 0≤α≤1, 0≤β≤1, 0≤γ≤1, 0≤δ≤0.4, 0≤z′≤0.4 and 0≤h′≤3.

16. The device of claim 13, wherein the anode comprises lithium metal, graphitic material, amorphous carbon, Li₄Ti₅O₁₂, tin alloy, silicon, silicon oxide, silicon mono-oxide, silicon alloy, intermetallic compound, or mixtures thereof.

17. The device of claim 13, wherein the anode is a composite anode comprising an active material silicon or silicon alloy and a conductive polymer coating around the active material.

18. The device of claim 17, wherein the conductive polymer is polyacrylonitrile (PAN).

19. The device of claim 13, wherein the device comprises a lithium battery, lithium-ion battery, lithium-sulfur battery, lithium-air battery, sodium ion battery, magnesium battery, lithium/MnO₂ battery, or Li/poly(carbon monofluoride) battery.

20. The device of claim 13, wherein the device comprises a capacitor or solar cell.

21. The device of claim 13, wherein the device comprises an electrochemical cell.

22. The device of claim 13, further comprising a porous separator separating the anode and cathode from each other.

23. The device of claim 22, wherein the porous separator comprises an electron beam-treated micro-porous polyolefin separator or a microporous polymer film comprising nylon, cellulose, nitrocellulose, polysulfone, polyacrylonitrile, polyvinylidene fluoride, polypropylene, polyethylene, polybutene, or co-polymers or blends of any two or more such polymers.

24. The device of claim 13, wherein the aprotic organic solvent system comprises an open-chain or cyclic carbonate, carboxylic acid ester, nitrite, ether, sulfone, ketone, lactone, dioxolane, glyme, crown ether, siloxane, phosphoric acid ester, phosphite, mono- or polyphosphazene or mixtures thereof.

25. The device of claim 13, wherein the aprotic organic solvent system is present in a concentration of from 60 wt. % to 90 wt. % in the formulation.

26. The device of claim 13, wherein the cation of the metal salt is an alkali metal.

27. The device of claim 26, wherein the alkali metal is lithium or sodium.

28. The device of claim 13, wherein the cation of the metal salt is aluminum or magnesium.

29. The device of claim 26, wherein the alkali metal salt is present in a concentration of from 10 wt. % to 30 wt. % in the formulation.

30. The device of claim 13, wherein the formulation further comprises at least one additive.

31. The device of claim 30, wherein the additive comprises a sulfur-containing compound, phosphorus-containing compound, boron-containing compound, silicon-containing compound, fluorine-containing compound, nitrogen-containing compound, compound containing at least one unsaturated carbon-carbon bond, carboxylic acid anhydride or the mixtures thereof.

32. The device of claim 30, wherein the additive is present in a concentration of from 0.01 wt. % to 10 wt. % in the electrolyte.
